(19) United States
(12) Patent Application Publication
(10) Pub. No.: US 2026/0205329 A1
(43) Pub. Date: Jul. 16, 2026

(54) ELECTRONIC TIMING CHANNEL READOUT WITH MULTIPLEXING CAPABILITIES

(71) Applicants: THE BOARD OF TRUSTEES OF THE LELAND STANFORD JUNIOR UNIVERSITY, Stanford, CA (US); LAWRENCE BERKELEY NATIONAL LABORATORY, Berkeley, CA (US)

(72) Inventors: Shirin POURASHRAF, Mountain View, CA (US); Joshua W. CATES, San Francisco, CA (US); Craig S. LEVIN, Palo Alto, CA (US)

(21) Appl. No.: 19/128,034

(22) PCT Filed: Nov. 8, 2023
(86) PCT No.: PCT/US2023/079151

§ 371 Date: May 7, 2025

Related U.S. Application Data
(60) Provisional application No. 63/382,824, filed on Nov. 8, 2022.
(60) Provisional application No. 63/479,783, filed on Jan. 13, 2023.

(51) Int. Cl. H04L 25/02; A61B 6/03; G01T 1/20; G01T 1/29

(52) U.S. Cl. CPC H04L 25/0272; A61B 6/037; G01T 1/2018; G01T 1/2985

(57) ABSTRACT

An electronic fast timing multiplexing readout for multiplexing a plurality of LVDS timing signals from a plurality of timing channels. First groups of balun transformers convert received LVDS timing signals from the timing channels to single-ended output voltage signals. One or more balun transformers convert the single ended output signals to one or more differential mode output signals. A comparator (digitizer) produces a multiplexed LVDS timing signal along a multiplexed output channel.

### CROSS-REFERENCE TO RELATED APPLICATIONS

This application claims priority to and the benefit of U.S. Provisional Patent Application No. 63/382,824, filed Nov. 8, 2022, and U.S. Provisional Patent Application No. 63/479,783, filed Jan. 13, 2023. Both applications are incorporated by reference herein in their entirety.

### FEDERALLY SPONSORED RESEARCH OR DEVELOPMENT

This invention was made with Government support under contracts CA009695, CA214669, and EB025125 awarded by the National Institutes of Health. The Government has certain rights in the invention.

### FIELD

This disclosure relates generally to electronic measurement systems. Embodiments of the invention relate more particularly to devices and systems for medical imaging such as Positron Emission Tomography (PET).

### BACKGROUND

Positron emission tomography (PET) is a diagnostic imaging modality that is commonly used to non-invasively visualize and quantify the molecular pathways of disease such as but not limited to cancer, neurological diseases, cardiovascular diseases, and others for detection, staging, and guiding/monitoring treatment. A PET study involves collection of millions of annihilation photon pairs being emitted from a positron-emitting radionuclide-labeled contrast agent injected into the patient. The position, energy, and arrival time of every (511 keV) photon is measured by the PET system.

To enhance PET capabilities in lesion detection and quantification for, as an example, earlier detection of primary and metastatic malignant lesions, it is desirable to improve the coincidence time resolution (CTR) of a PET system. This can advance time-of-flight (TOF) performance, which has substantial benefits for reconstructed image signal-to-noise ratio (SNR). Benefits include substantially better lesion detection, and/or much lower injected radiation dose or study duration.

Recent positron emission tomography (PET) imaging systems developed in the field of medical imaging use a large number of silicon photomultipliers (SiPMs) for high resolution and excellent TOF performance. Therefore, effective and scalable multiplexing readout approaches are demanded to reduce the number of electronic channels. Unfortunately, multiplexing methodologies generally degrade the fast timing capabilities necessary for TOF-PET.

### SUMMARY

According to one aspect of the disclosed embodiments, an electronic fast timing multiplexing readout is provided for multiplexing low voltage differential signaling (LVDS) timing signals. The readout comprises a first set of balun transformers respectively coupled to timing channels and configured to receive LVDS timing signals and convert the received LVDS timing signals to single-ended voltage signals. One or more second balun transformers are configured to convert the single ended voltage signals from the first balun transformers to differential mode output signals. A high speed comparator (e.g., a digitizer) is configured to produce a multiplexed LVDS timing signal along a multiplexed output channel.

According to another aspect, a detector of emitted photons comprises a readout as provided herein; detectors for receiving photons emitted from a patient and to generate timing and energy signals in response, the detectors being arranged in detector arrays; and a plurality of timing channels, each being respectively coupled or related to one of the detector arrays and generating one of the plurality of LVDS timing signals. According to another aspect, an imaging system comprises one or more detector units comprising the detector; processing electronics configured to receive the multiplexed LVDS timing signal from the multiplexing readout and to receive generated energy signals from the detectors; and a processor configured to generate an image by processing the received signals.

Example processing electronics include but are not limited to a field programmable gate array (FPGA), an application specific integrated circuit (ASIC), or others. Example processors include, but are not limited to, computer processors, which computer processors may operate with a memory.

According to another aspect, a method for multiplexing a plurality of LVDS fast timing signals from a plurality of timing channels comprises, for each of the timing channels, receiving the LVDS fast timing signals from the respective timing channel and converting the received LVDS fast timing signals to a single-ended voltage signal; converting the single ended voltage signals to differential mode voltage signals; hardwiring the differential mode voltage signals; and digitizing the hardwired differential mode voltage signals using a final high speed comparator to produce a multiplexed LVDS timing signal along a multiplexed output channel.

### DESCRIPTION OF THE DRAWINGS

The drawings set forth herein are illustrative of exemplary embodiments provided herein and are not meant to limit the scope of the invention as encompassed by the claims.

The present disclosure will become more fully understood from the detailed description and the accompanying drawings, wherein:

FIG. 1 shows an example side-readout time-of-flight PET (TOF-PET) system according to an example embodiment.

FIG. 2A shows a scalable TOF-PET system including detector modules according to an example embodiment.

FIG. 2B shows an example detector layer unit with an electronic readout and data acquisition block according to an example embodiment.

FIG. 3 shows a block diagram for an example 100 ps CTR TOF-PET detector unit multiplexing readout according to an example embodiment.

FIG. 4 shows a block diagram for an example electronic timing channel and a timing multiplexer for the readout of FIG. 3.

FIG. 5 shows example compact printed circuit boards (PCB) for implementing a readout.

FIG. 6 shows example readout components.

FIG. 7 shows an example set-up used in experiments.

FIG. 8 shows an example of CTR performance of two coincident detector layer units, each with 24:1 ratio multiplexed timing signals.

FIG. 9A shows an example scalable side-coupled detector layer unit; FIG. 9B shows an example scalable sub-module unit; and FIG. 9C shows an example scalable full detector module design for providing an example scalable TOF-PET system.

FIG. 10 shows another example multiplexing timing readout with a reduced number of balun transformers.

### DESCRIPTION

Embodiments herein provide, among other things, an electronic multiplexing readout that can receive and multiplex multiple LVDS timing channels for very fast (e.g., order of picoseconds) timing signals. In an example method, an 8:1 timing multiplexing ratio is applied per detector layer, though other multiplexing ratios are possible. Prior to the present disclosure, no high performance conventional electronic components were available to combine such sharp-edge LVDS timing channels and reduce them to only one output.

An example multiplexing readout multiplexes 8 LVDS timings, each from a plurality of fast timing sensors such as from a plurality of (e.g., three) silicon photomultipliers (SiPM), to provide a 24:1 multiplexed timing signal. An example energy signal multiplexing method can provide an effective design to extract, as a nonlimiting example, 3 mm depth-of-interaction (DOI) information through the entire large area detector modules. The multiplexed timing signal can be output for downstream processing for time-to-digital conversion (TDC).

In an example multiplexing operation, each of the eight LVDS timings is converted to a single ended output voltage using a high frequency (e.g., 0.4-1.8 GHZ) balun transformer. The output of the balun transformer is AC-coupled to the input of another balun transformer, which is used to convert this single-ended signal to a differential mode output, which can then be coupled to the other (e.g., seven) differential timing outputs at nodes inputting the differential pins of a comparator to produce a final LVDS timing channel.

Multiplexing in example methods and systems can reduce number, size, and/or complexity of electronic readout channels, without degrading timing resolution performance. Resulting systems can also be more efficient, e.g., having reduced power consumption. Example embodiments described herein for illustrating inventive aspects provide, among other things, a readout which can be made very compact and scalable to multiplex, for example, 24 fast outputs of each 6×4 SiPM array (or 8 LVDS timing signals) to only 1 LVDS timing channel per detector layer unit while maintaining high performance, e.g., ~100 ps CTR. These are believed to be the first multiplexing circuits in the art with this high level of multiplexing (24:1 or even higher multiplexing) that do not significantly degrade the timing resolution performance.

Using example timing channel multiplexing approaches herein, an example embodiment can provide a 100 ps coincidence time resolution (CTR) TOF-PET scanner to constrain events to, for instance, a 1.5 cm segment along each LOR, which is believed to be a higher CTR TOF-PET than previously known in the art. Such a system can provide further enhancements in reconstructed image SNR, which, if desired, may be exploited to better visualize and quantify disease or, alternately, to reduce scan time or dose injected into the patient, e.g., by enabling >1.5-fold SNR improvement relative to currently available TOF-PET scanners.

A nonlimiting example multiplexing electronic readout system and method can be embodied or implemented in full ~5×5 cm² TOF-PET modules each including 64 detector layer units. These modules, for instance, can be assembled in a TOF-PET ring achieving a ~100 ps CTR TOF-PET system. The TOF-PET system can also be (but need not be) combined with an x-ray CT scanner to provide multiple mode imaging. A nonlimiting example implementation includes a 16-module partial-ring prototype 100 ps CTR TOF-PET system butted against a stand-alone CT scanner.

Preferred embodiments will now be discussed with respect to the drawings. The drawings include schematic figures that are not to scale, which will be fully understood by skilled artisans with reference to the accompanying description. Features may be exaggerated for purposes of illustration. From the preferred embodiments, artisans will recognize additional features and broader aspects of the invention.

Excellent coincidence time resolution (CTR) is essential in high performance time of flight positron emission tomography (TOF-PET) imaging (e.g., see J. W. Cates, et al., “Evaluation of a clinical TOF-PET detector design that achieves <100 ps coincidence time resolution”, Phys. Med. Biol., 2018). For illustration, a general comparison of conventional PET and TOF-PET will be discussed.

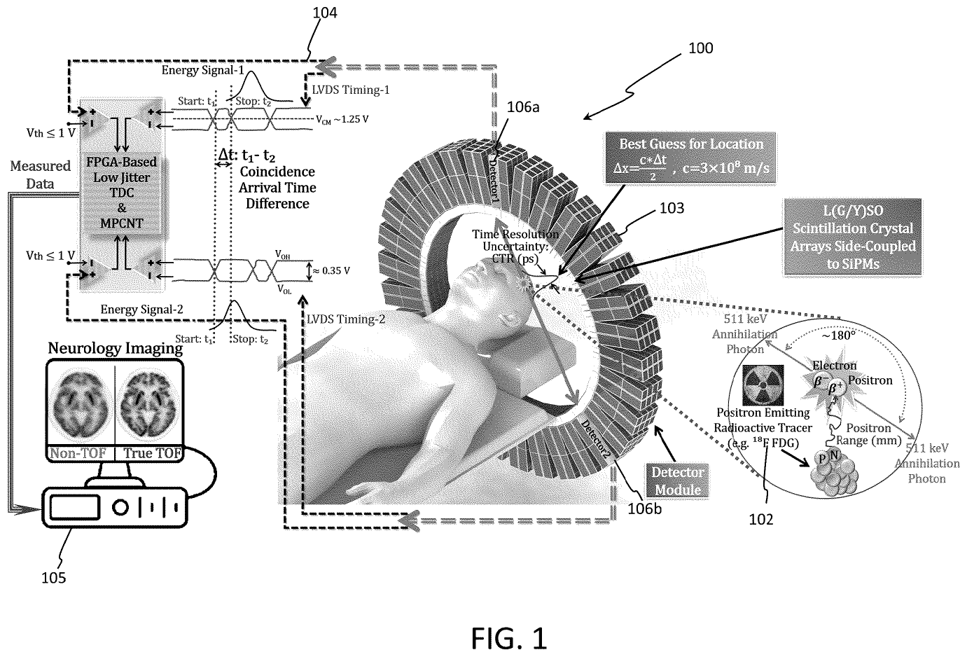

FIG. 1 shows an example side-readout time-of-flight PET (TOF-PET) scheme 100. In an example PET imaging method, a chemical molecule may be labeled with a positron emitting isotope to provide a PET tracer 102. Example positron emitter radionuclides include but are not limited to ¹¹C, ¹³N, ¹⁵O, and ¹⁸F. The PET tracer may be injected into a subject. The positron emitting isotope emits positrons that collide with electrons in annihilation events. The annihilation event produces a pair of gamma ray photons (e.g., 511 keV photons) that are emitted in opposite directions along a line of response (LOR). The emitted photons may be detected by detectors in a ring of detector modules 103 surrounding the subject. Example detector modules can include one or more scintillation crystals and photosensors, and an electronic readout 104. For example, each emitted photon may be received and detected by a detector within the ring of detector modules. The detector modules produce data via output signals that may be processed by a processor such as but not limited to a computer 105. The processor performs image processing and data analysis to generate one or more PET images. Image processing and data analysis may include predicting a location of the annihilation event (an event localization) along a line of response (LOR).

In a conventional PET method, for a pair of detected photons, an equal probability for an annihilation event location may be provided for all voxels along the LOR; e.g., Δx>D, where D may be a patient diameter (as a nonlimiting example, 40 cm). In a time-of-flight PET (TOF-PET) scheme, arrival times t₁, t₂ are determined for each of the pair of photons at their respective detectors, e.g., detectors 106a, 106b. Δt=t₂−t₁ is the coincidence arrival time difference. A coincidence time resolution (CTR) may be expressed as the full-width-at-half-maximum (FWHM) of the Δt distribution. A localization error can be expressed as

$$Δ x = \frac{C T R}{2} \times c ,$$

where c is the speed of light, e.g., c=3×10¹⁰ cm/s. For a patient diameter D the probability for the location of the annihilation event in TOF-PET can be confined to a relatively small segment on the LOR, e.g., Δx<D, and

$$Δ x = \frac{C T R}{2} \times c .$$

A TOF-PET image can be equivalent to a non-TOF image with a larger number of counts. Alternatively or additionally, TOF reconstruction can operate as virtual counts or as a sensitivity amplifier for a PET scanner. Each coincidence event in TOF-PET can yield more information, e.g., by a factor of

$$\sqrt{\frac{D}{Δ d}} .$$

Improving the coincidence time resolution (CTR) can thus improve resolution and reconstructed image signal-to-noise ratio (SNR) in TOF-PET, e.g.,

$$S N {R}_{T O F} = \sqrt{\frac{D}{\frac{CTR}{2} \times c}} \cdot {SNR}_{non - TOF} , where \sqrt{\frac{D}{\frac{CTR}{2} \times c}}$$

may be referred to as gain. For instance, a CTR of 1000 ps can provide a localization of 15 cm and a gain of 1.6. As another example, a current state-of-the-art CTR TOF-PET/CT system provides a CTR of 214 ps and a localization along an LOR of 3.7 cm. A CTR of 100 ps, by contrast, can provide a localization of 1.5 cm and a gain of 5.2.

Improving CTR in TOF-PET can in turn improve reconstructed image SNR, signal-to-background ratio, image quality, and/or accuracy. Alternatively or additionally, for the same image quality as provided in conventional PET, an improved CTR can allow for a lower injected tracer dosage to patients and/or a shorter scan time. A wider category of patients may also be served. In example medical imaging applications, improved CTR for TOF-PET methods can provide benefits such as, but not limited to, improved lesion detectability (e.g., via a higher SNR) and/or better lesion quantification (e.g., SUV precision and accuracy), and may provide a more faithful representation of cancer biomarker distribution (e.g., better visualization of tumor heterogeneity).

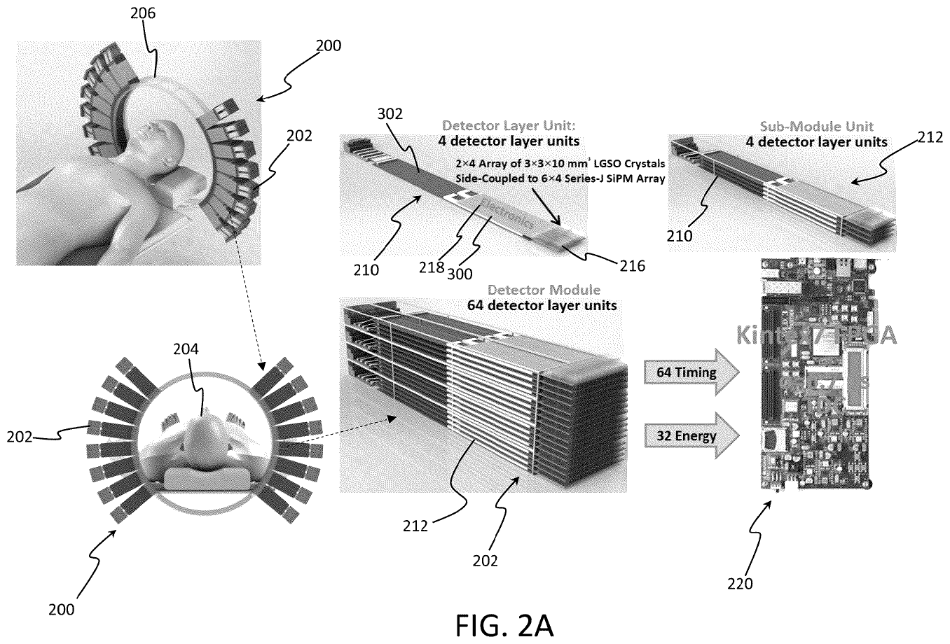

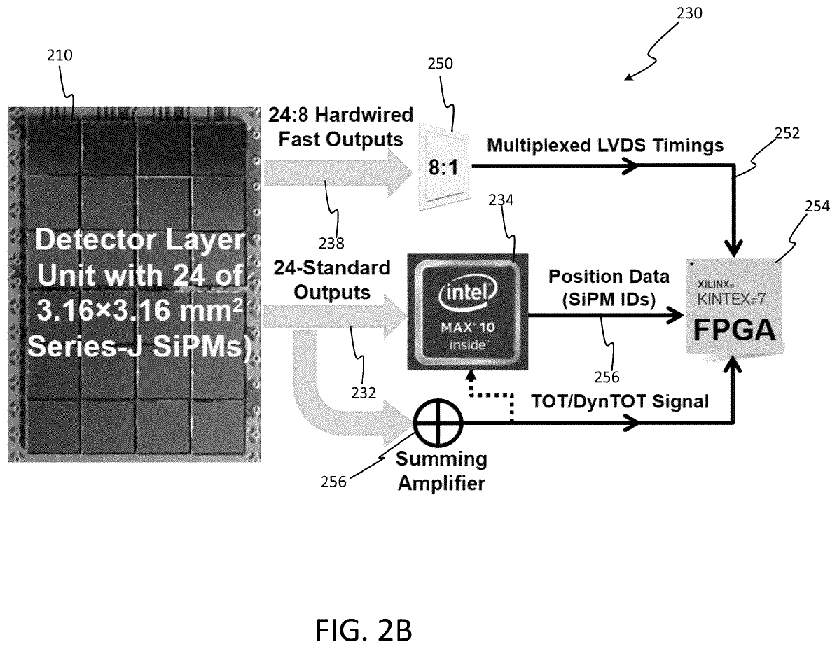

An example scalable TOF-PET system 200 is shown in FIG. 2A. The system 200 includes (e.g., 16) detector modules 202, which in the example system are disposed in a partial ring around a subject 204. The detector modules 202 may be, but need not be, butted against a scanner such as but not limited to a stand-alone CT scanner 206.

Each example detector module 202 includes a plurality of detector layer units 210, a nonlimiting example being 64 detector layer units. For instance, each example detector module 202 may include a plurality of sub-module units 212 having a plurality of (e.g., four) detector layer units 210. Each detector layer unit 210 includes an array of scintillation crystals (e.g., LGSO crystals) 216 coupled (e.g., side-coupled) to a photodetector array (e.g., silicon photomultipliers (SiPMs)) along with processing electronics 218. An FPGA-based (e.g., Kintex-7 FPGA) time to digital converter (TDC) 220 processes timing signals and energy signals.

An electronic readout and data acquisition block 230 for the detector layer unit 210 is shown generally in FIG. 2B. The example electronic readout and data acquisition block 230 can be operated to measure the 3D position of 511 keV photon interactions in a scintillation detector array, e.g., through individually connecting the standard outputs 232 of each of 24 SiPMs (anode signals) to a processing device such as a complex programmable logic device (CPLD) 234 (e.g., M08SCM153C8G, Intel). An example detector layer unit 210 includes twenty-four 3.16×3.16 mm² Series-J SiPMs.

Each three SiPM fast outputs are hardwired together, providing a 24:8 multiplexing ratio. The hardwired fast outputs 238 are provided to a multiplexer 250 which may be embodied in an 8:1 multiplexed low voltage differential signaling (LVDS) timing multiplexer, via eight timing channels or timing chains, as disclosed in further detail herein. The LVDS timings 252 are provided to an FPGA 254, along with position data (e.g., SiPM IDs) 256 from the CPLD 234. A summing operational amplifier 258 (e.g., ADA4817, Analog Devices) can also be used to produce the total energy per detector unit which can be processed by the FPGA 254 using time over threshold (TOT) or dynamic TOT methods. The latter linearizes the shaped energy and can be implemented, e.g., using discrete off-the-shelf electronic components or custom components (or a combination).

A TOF-PET detector has exhibited an approximately 100 ps FWHM CTR using a low jitter field programmable gate array (FPGA)-based time to digital converter (TDC). S. Pourashraf, et al., “Scalable electronic readout design for a 100 ps coincidence time resolution TOF-PET system,” Phys. Med. Biol., 2021; and S. Pourashraf, et al, “Investigation of Electronic Signal Processing Chains for a Prototype TOF-PET System with 100 ps Coincidence Time Resolution”, IEEE TRPMS, 2022, incorporated by reference in their entirety herein, disclose a TOF-PET detector readout providing a roughly 100 ps full-width-at-half-maximum (FWHM) CTR using a low jitter FPGA-based TDC. This 100 ps CTR implies an additional >1.5-fold improvement in reconstructed image signal to noise ratio (SNR), as compared with the best CTR currently achieved for clinical TOF-PET systems. The results disclosed in S. Pourashraf, et al., 2021 and in S. Pourashraf, et al., 2022 were achieved for one low voltage differential signaling (LVDS) timing channel using single 3×3×10 mm³ scintillation crystals side-coupled to a 6×4 linear array of three 3×3 mm² SiPMs while their fast outputs were hardwired together, creating a 24:8 multiplexing ratio.

The limited numbers of TDC channels in the FPGA 254 and the necessity of maintaining a high packing fraction in a detector system provides a need to highly multiplex the timing channels. Multiplexing the timing channels can facilitate a scaling-up process while saving resources. However, standard LVDS signals are current mode drivers and cannot simply be combined together. Simply hardwiring SiPMs signals together can introduce parasitic capacitance, leading to CTR degradation. Multiplexing strategies such as resistive charge division lead to a high RC constant and CTR degradation. Delay-line methods provide lower SNR, especially for longer delays, and can require additional resources from the FPGA.

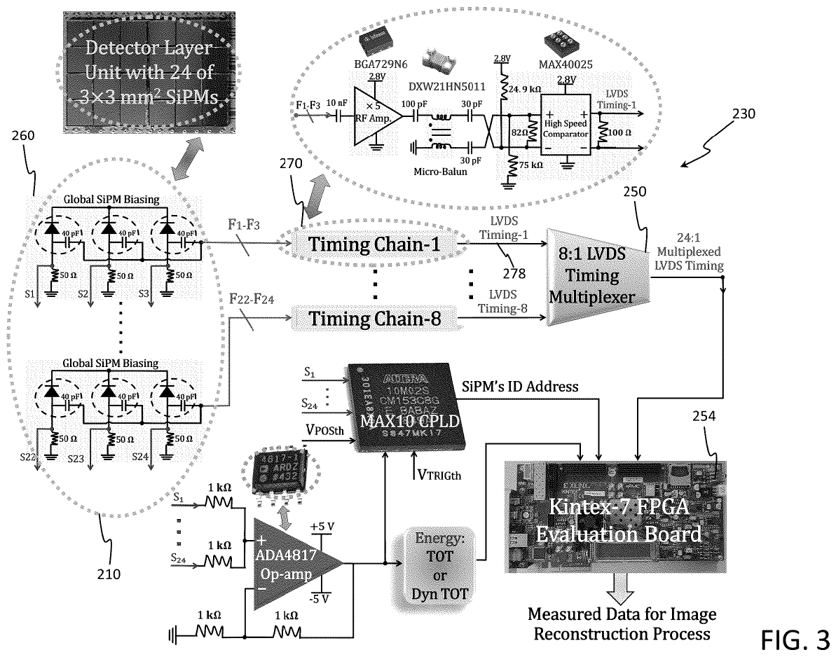

FIG. 3 shows features of the electronic readout and data acquisition block 230 for an example ~100 ps TOF-PET detector layer unit 210 with SiPMs and the LVDS timing multiplexer 250 according to an example embodiment. The example detector layer unit 210 includes a 6×4 array of SiPMs 260 (for example, as provided by On Semiconductor MicroFJ-30035), where each three SiPMs are hardwired for 8 LVDS timing channels 270.

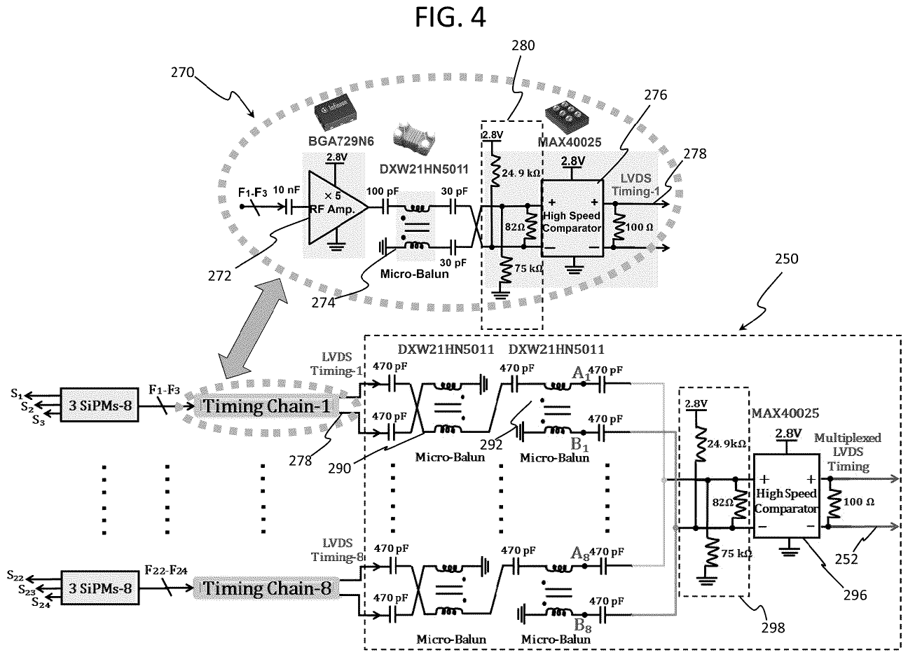

FIG. 4 shows components of an example electronic timing chain or timing channel (timing channel) 270 for one of the multiple timing channels and of the LVDS timing multiplexer 250. The timing channel 270 includes an RF amplifier 272, a balun transformer (micro-balun) 274, and a digitizing stage (comparator) 276 that outputs an LVDS timing 278. An example timing channel 270 may be, but need not be, configured generally similarly to the timing electronic chain disclosed in S. Pourashraf, et al., “Scalable electronic readout design for a 100 ps coincidence time resolution TOF-PET system,” Phys. Med. Biol., 2021. To optimize footprint and power consumption, the RF amplifiers 272, the balun transformers 274, and the digitizing stage (comparator) 276 can be (but need not be) provided by low noise/smaller counterparts such as but not limited to BGA729N6, DXW21HN5011, and MAX40025, respectively, as shown in FIG. 4.

An external fail-safe resistive biasing 280 may be provided at the differential inputs of the comparator 276. The example resistive biasing 280 can provide (e.g., 2.1V) common mode voltages for the comparator's 276 positive and negative inputs.

FIG. 4 further illustrates features of the LVDS timing multiplexer 250. The example LVDS timing multiplexer 250 is embodied in a novel multiplexing timing readout that can address the challenge of combining LVDS timing channels that are current mode drivers. In the example readout, each of the (as shown, eight) LVDS timings 278 is first converted to a single ended output using a first high frequency balun transformer 290, e.g., embodied in passive micro-baluns (a nonlimiting example being DXW21HN5011). The output of the balun transformer is AC-coupled to an additional or second balun transformer 292, e.g., embodied in passive micro-baluns (a nonlimiting example being DXW21HN5011), that is used to convert this single-ended signal to a differential signal (differential mode output or differential timing output).

The differential signals are then hardwired to the other (e.g., 7) differential timing outputs at nodes A_{1 . . . 8} & B_{1 . . . 8} inputting the differential pins of a last comparator (digitizer) (e.g., MAX40025) 296 to produce a final LVDS timing channel 252. An external fail-safe resistive biasing 298 may also be provided at the differential inputs of the last comparator 296. An example resistive biasing 298 can provide, as a nonlimiting example, V_{id}=−2.5 mV dropped on (e.g., 82Ω) line termination resistors to clean up LVDS timing signals at idle line states. An example readout system 230 can provide V_{id}<2.5, as the noise level can be very low (e.g., ~1 mV).

An example multiplexing readout 230 for TOF-PET can be implemented by adding passive balun transformers (micro-baluns), e.g., first and second passive baluns 290, 292 and an additional comparator, e.g., comparator 296 to the timing electronic chain 270, without the need for additional components. This can provide simplicity, a lower implementation cost, a small footprint, and power efficiency (as a nonlimiting example, 4.5 mW/channel additional power dissipation), among other advantages.

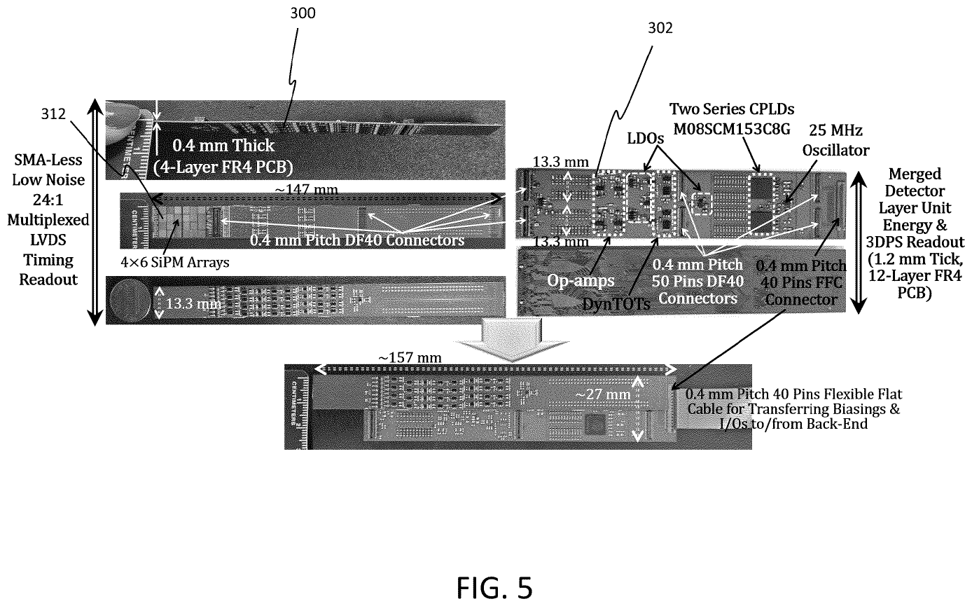

Very low noise multi-layer printed circuit boards (PCBs) embodied in PCBs 300, 302 can be provided for implementing readout electronics, as shown by example in FIG. 5. The PCBs 300, 302 can be configured to be compact and scalable. The example PCBs may include a board 300 embodied in a four-layer FR4 PCB in which main circuitry (effective regions) for a timing readout is implemented with a limited width (e.g., 13.3 mm). A 4×6 array of 3×3 mm² SiPMs 312, for instance can be provided on the PCB 300. The PCB 300 can further include outputs for multiplexed LVDS timing. The PCB 302 may, for instance, process energy and position signals, and may be embodied in a 12-layer FR4 PCB board in which main circuitry (effective regions) for energy and 3DPS readout can be implemented in a limited width of 13.3 mm. Incorporating such effective regions into a limited width allows all components to fit behind the 4×6 SiPM array in the example detector design.

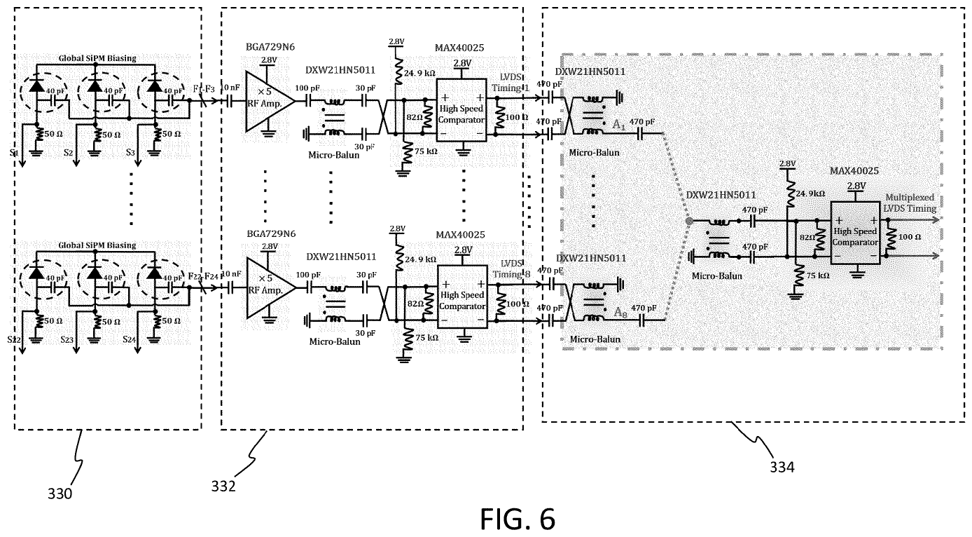

FIG. 6 shows example readout electronics that may be implemented, e.g., in a circuit board, including a combined SiPM array 330, timing channels 332, and a timing multiplexing chain 334 according to an example embodiment. The example multiplexing chain 334 may be implemented in an example board, e.g., without the need to increase the (e.g., 13.3 mm) width, and high sensitivity can still be provided. Including the example multiplexing chain 334 may, but need not, increase a length of the first PCB board 300, e.g., by a small amount (a nonlimiting example being 27 mm). Since the example timing signals may be, for instance, already digitized, the increased length should not increase jitter.

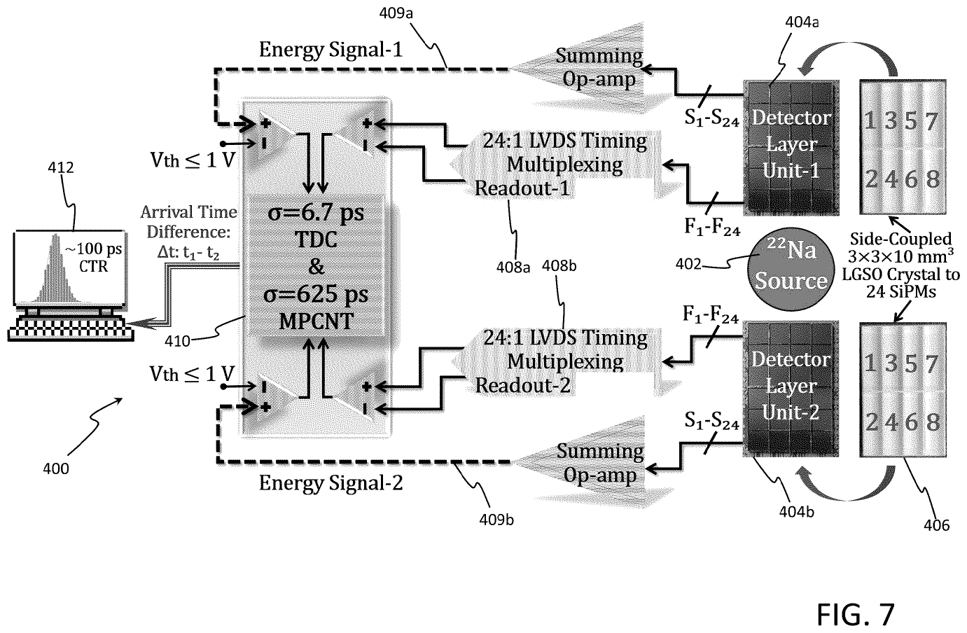

FIG. 7 shows an experimental coincidence set-up 400 used to evaluate CTR in experiments. A (~8 μCi) ²²Na point-source 402 was placed between two TOF-PET detector layer units 404a, 404b, each including arrays 406 of 3×3×10 mm³ LGSO: Ce scintillation crystals (Oxide, Yamanashi, Japan) coated with BaSO₄ reflector and side-coupled to SiPMs using optical grease (BC-360, Saint-Gobain). For data acquisition, 24:1 ratio multiplexed timing signals from LVDS timing multiplexing readouts 408a, 408b were connected to a low jitter (σ=6.7 ps) FPGA-based TDC 410. Energy signals 409a, 409b from the detector layer units 404a, 404b were generated. A processor provided by a computing device 412 processed the collected data.

Approximately 12000 coincident events were collected for each CTR experimental dataset in an example. For further improving CTR performance, the SiPMs were biased at V_{SIPM}=31.0 V and the optimal voltage supplies of both RF amplifiers and LVDS comparators were set at V_{RF}=V_{Comp}=2.8 V. To clean up the LVDS timing signals at idle line states where there is no triggering signal coming from SiPMs, a fail-safe resistive biasing was carefully applied at the differential inputs of each MAX40025 comparator, such as the resistive biasing 280, 298 shown in FIG. 4. This example resistive network generated around 2.1 V common mode voltages at the comparator's positive and negative inputs accordingly with a voltage difference (small offset) of about V_{id}=−2.5 mV dropped on 82Ω line termination resistors. An average coincident timing performance of 107.0±3.6 ps FWHM was achieved after combining 8 LVDS timing channels (24 SiPMs' fast outputs) per detector unit and using 2×4 arrays of 3×3×10 mm³ LGSO crystals side-coupled to 6×4 arrays of SiPMs. No cooling system was used during the experiments.

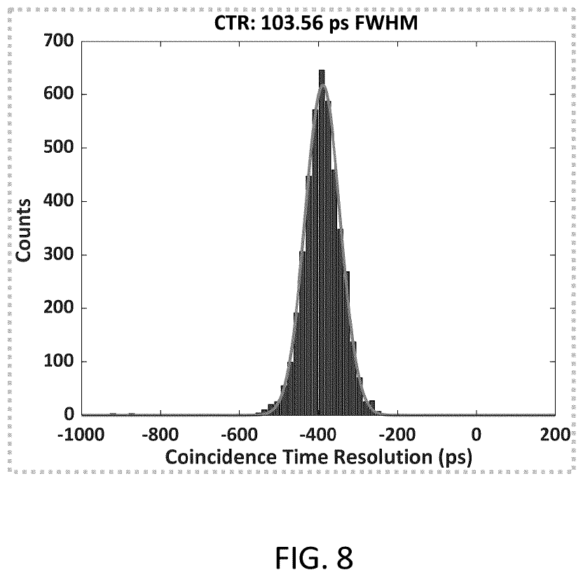

FIG. 8 depicts coincidence time difference spectra for example coincidence detector layer units. The example results show near 100 ps CTR over multiple measurements using a combined 8-timing channel (24 SiPM's fast output). This is believed to be the first demonstration of 100 ps FWHM coincidence resolving time between TOF-PET detectors having a 24:1 timing multiplexing ratio.

The results illustrate that by using low noise, high speed, power efficient, and compact electronic components, an example electronic design approach can be used to build an electronic readout for TOF-PET detector layer unit, multiplexing 24 fast timing signals to only one LVDS timing channel while maintaining ~100 ps CTR performance. These beneficial CTR results were achieved even with the presence of other non-ideal factors such as light sharing between crystals in the array, inter-channel timing skew, multiplexing challenges, and without the implementation of an optional cooling system to compensate for the temperature variations that can affect the CTR performance.

In addition to excellent coincidence timing performance, the compact board width of an example multiplexing readout facilitates the scalability of example high sensitivity TOF-PET detector modules. Example detector layer units can be provided, for instance, in a fully implemented digital architecture, including channel-dense FPGA-based TDCs.

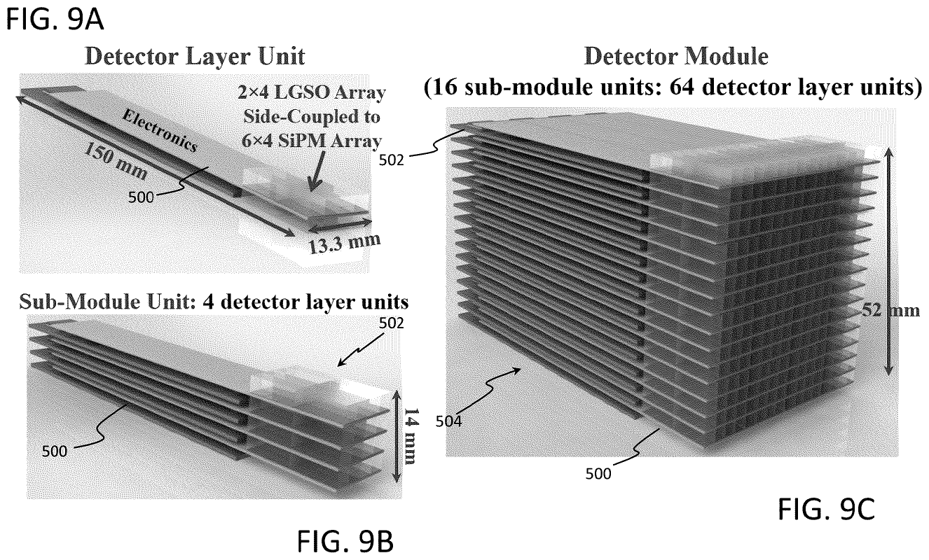

FIGS. 9A-9C show an example scalable detector layer unit system for providing 100 ps CTR TOF-PET, where FIG. 9A shows an individual side-coupled detector layer unit 500, FIG. 9B shows a sub-module unit 502 including four detector layer units 500, and FIG. 9C shows a detector module 504 including 16 sub-modules and 64 detector layer units. The example TOF-PET detector design can be scaled up to, as a nonlimiting example, a full ~5×5 cm² detector module such as detector module 504 including 64 detector layer units 500 (16 sub-module units 502). After multiplexing signals, an example scanner can include, for instance, 64 timing channels per detector module 504.

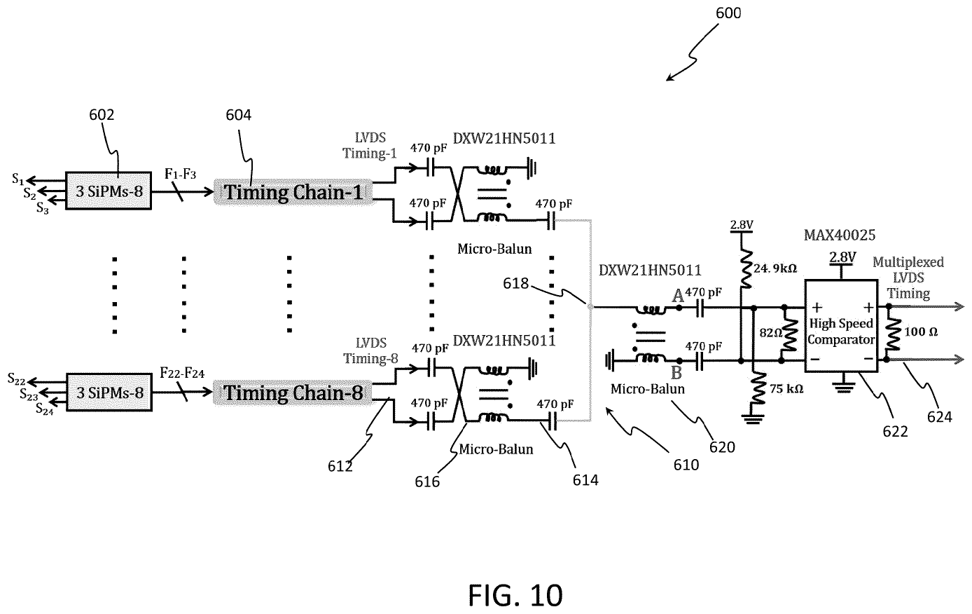

FIG. 10 shows another example readout 600. The multiplexing timing readout 600 in FIG. 10 includes an SiPM array 602 and timing channels 604, which can be (but need not be) configured generally similarly to the timing channel 270 in FIGS. 3 and 4. The readout 600 further includes a timing multiplexer 610 in which, after converting (e.g., 8) LVDS timings 612 to (e.g., 8) single ended voltage outputs 614 using first high frequency balun transformers (e.g., micro-baluns) 616, they can be all hardwired together, e.g., at 618. Then, a single (common) high frequency balun transformer (e.g., micro-balun) 620 can be provided as a second balun transformer to convert this combined single-ended signal to a differential mode output inputting the differential pins of the last (e.g., MAX40025) comparator 622 to produce a final multiplexed LVDS timing channel 624. The example multiplexing timing readout 600 in FIG. 10 can involve fewer numbers of balun transformers (e.g., nine versus sixteen), which can reduce a footprint while providing similar or comparable multiplexing performance. Alternatively or additionally, the example multiplexing approach exemplified in FIG. 10 may be used to serve additional (e.g., more than 24) SiPMs, a nonlimiting example being 48 SiPMs.

Although example features of timing channels and timing multiplexers are described herein for illustration, example multiplexers may also be implemented using, as nonlimiting examples, one or more features of the PET readout timing channels disclosed in Pourashraf, et al., “Scalable electronic readout design for a 100 ps coincidence time resolution TOF-PET system,” Phys. Med. Biol. 66, 2021; and S. Pourashraf, et al, “Investigation of Electronic Signal Processing Chains for a Prototype TOF-PET System with 100 ps Coincidence Time Resolution”, IEEE TRPMS, Vol. 6, No. 6, 2022.

It will be appreciated that inventive principles provided herein can also be applied to other n×m SiPM arrays and to other numbers of timing signals and fast outputs, and example detector layer units can be configured to provide multiplexing levels greater than or less than 24:1. For example, multiplexing readouts can serve a larger array of SiPMs (e.g., more than 24, nonlimiting examples being 48, or between 24 and 48). For instance, additional balun transformers may be included to convert more than 8 LVDS timing signals (e.g., each related to three combined (e.g., hardwired) SiPMs' fast outputs, such as provided in examples herein) to the differential voltage signals, and these may be combined (e.g., hardwired) through a multiplexing scheme and sent to a final comparator. As another example, instead of three fast outputs (e.g., timing signals) being hardwired, two fast outputs (timing signals) or greater than three fast outputs may be hardwired. Example embodiments can enable 100 picosecond (ps) coincidence time resolution (CTR), which hereto has not been possible in a CTR TOF-PET system, though it is also possible to provide lower CTR resolutions.

Example multiplexers can be used to reduce electronic readout channels, thus reducing required resources, power consumption, and complexity. Specific components of the multiplexer can vary while still providing good performance (e.g., fast timing, such as on the order of picoseconds).

Example multiplexers can be implemented generally in applications using multiple timing sensors, especially where good time resolution is critical. Examples include but are not limited to imaging, autonomous vehicles, object detection, active pedestrian safety, robot navigation, people or item counting, etc. Fast timing sensors may include, but are not limited to, photosensors, imaging sensors, vehicle sensors, object sensors, or navigational sensors. As a nonlimiting example, for medical imaging modalities, time of flight positron emission tomography (TOF-PET) scanners for industry and/or research can benefit by employing example approaches herein.

Example embodiments provide, among other things, an electronic fast timing multiplexing readout for multiplexing a plurality of LVDS timing signals from a plurality of timing channels, the readout comprising: a first group of balun transformers being respectively fed by each of the LVDS timing channels, each of the balun transformers in the first group being configured to receive LVDS timing signals from the respective timing channel and convert the received LVDS timing signal to a single-ended output voltage signal; and one or more second balun transformers configured to convert the single ended output signals from the first group of balun transformers to differential mode output signals; and a comparator or digitizer configured to receive the differential mode output signals and produce a multiplexed differential timing signal along a multiplexed output channel. In addition to any or all of the above features in this paragraph, for each of the timing channels the received LVDS timing signals may be for a plurality of fast outputs from a set of coupled fast timing sensors. In addition to any or all of the above features in this paragraph, the plurality of fast outputs may be hardwired. In addition to any or all of the above features in this paragraph, each of the timing channels may comprise: a balun transformer for receiving the plurality of hardwired fast outputs; and a digitizing stage coupled to said balun transformer and configured to output the LVDS timing signal. In addition to any or all of the above features in this paragraph, each of the timing channels may further comprise: an amplifier upstream of the balun transformer for amplifying the hardwired fast outputs that are received by the balun transformer. In addition to any or all of the above features in this paragraph, the fast outputs in each of the timing channels may comprise 2-3 fast outputs. In addition to any or all of the above features in this paragraph, the timing channels may comprise at least 2 timing channels. In addition to any or all of the above features in this paragraph, the timing channels may comprise at least 8 timing channels. In addition to any or all of the above features in this paragraph, the output of electronic timing chains may comprise or be low voltage differential signals (LVDS). In addition to any or all of the above features in this paragraph, the first balun transformers may be passive. In addition to any or all of the above features in this paragraph, each of the first balun transformers may be coupled to a different one of the second balun transformers. In addition to any or all of the above features in this paragraph, the first balun transformers may be coupled to a common second balun transformer. In addition to any or all of the above features in this paragraph, the fast timing sensors may be on the order of picoseconds. In addition to any or all of the above features in this paragraph, the fast timing sensors may comprise a photosensor. In addition to any or all of the above features in this paragraph, the fast timing sensors may comprise imaging sensors, vehicle sensors, object sensors, or navigational sensors. In addition to any or all of the above features in this paragraph, the multiplexed output channel may be coupled to a time-to-digital converter.

Other example embodiments provide, among other things, a detector of emitted photons comprising: a readout according to any or all of the features of the preceding paragraph; detectors for receiving photons emitted from a patient and to generate timing and energy signals in response, the detectors being arranged in detector arrays; and a plurality of timing channels, each being respectively coupled to one of the detector arrays and generating one of the LVDS timing signals. In addition to any or all of the above features in this paragraph, each of the detectors may comprise: a scintillator configured to receive photons emitted from a patient; and a photosensor configured to receive scintillation light from the scintillator in response to the scintillator receiving and to generate timing and energy signals in response.

Other example embodiments provide, among other things, an imaging system comprising: one or more detector units, each detector unit comprising the detector having any or all of the features of the preceding two paragraphs; processing electronics configured to receive multiplexed timing signals from the readout and to receive the energy signals from the one or more detector units; and a processor configured to generate an image by processing the received signals. In addition to any or all of the above features in this paragraph, the photons emitted from the patient may be 511 keV photons.

Other example embodiments provide, among other things, a method for multiplexing a plurality of LVDS fast timing signals from a plurality of timing channels, the method comprising: for each of the timing channels, receiving the LVDS fast timing signals from the respective timing channel and converting the received LVDS fast timing signals to a single-ended output signal; converting the single ended output signals to one or more differential mode output signals; and digitizing the combined differential mode outputs to produce a multiplexed LVDS timing signal along a multiplexed output channel. Example methods may further include any or all features from the above three paragraphs. In addition to any or all of the above features in this paragraph, the method may further comprise: receiving, by each of the plurality of timing channels, a plurality of fast outputs from a set of coupled fast timing sensors; amplifying the received fast outputs using a low noise RF amplifier; transforming the amplified fast outputs to differential signals using a balun transformer; and digitizing the transformed fast outputs to generate the LVDS timing signals for the respective timing channel.

### General

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition.

The entirety of each patent, patent application, publication and document referenced herein is hereby incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

A number of embodiments of the invention have been described. Nevertheless, it can be understood that various modifications may be made without departing from the spirit and scope of the invention. Accordingly, other embodiments are within the scope of the following claims.

What is claimed is:

1. An electronic fast timing multiplexing readout for multiplexing a plurality of LVDS timing signals from a plurality of timing channels, the readout comprising:
a first group of balun transformers being respectively fed by each of the LVDS timing channels, each of the balun transformers in the first group being configured to receive LVDS timing signals from the respective timing channel and convert the received LVDS timing signal to a single-ended output voltage signal; and
one or more second balun transformers configured to convert the single ended output signals from the first group of balun transformers to differential mode output signals; and
a comparator or digitizer configured to receive the differential mode output signals and produce a multiplexed differential timing signal along a multiplexed output channel.

2. The readout of claim 1, wherein for each of the timing channels the received LVDS timing signals are for a plurality of fast outputs from a set of coupled fast timing sensors.

3. The readout of claim 2, wherein the plurality of fast outputs are hardwired.

4. The readout of claim 1, wherein each of the timing channels comprises:
a balun transformer for receiving the plurality of hardwired fast outputs; and
a digitizing stage coupled to said balun transformer and configured to output the LVDS timing signal.

5. The readout of claim 4, wherein each of the timing channels further comprises:
an amplifier upstream of the balun transformer for amplifying the hardwired fast outputs that are received by the balun transformer.

6. The readout of claim 5, wherein the fast outputs in each of the timing channels comprises 2-3 fast outputs.

7. The readout of claim 1, wherein the timing channels comprise at least 2 timing channels.

8. The readout of claim 1, wherein the output of electronic timing chains are low voltage differential signals (LVDS).

9. The readout of claim 1, wherein the first balun transformers are passive.

10. The readout of claim 1, wherein each of the first balun transformers is coupled to a different one of the second balun transformers.

11. The readout of claim 1, wherein the first balun transformers are coupled to a common second balun transformer.

12. The readout of claim 1, wherein the fast timing sensors are on the order of picoseconds.

13. The readout of claim 1, wherein the fast timing sensors comprise a photosensor, imaging sensors, vehicle sensors, object sensors, or navigational sensors.

14. The readout of claim 1, wherein the multiplexed output channel is coupled to a time-to-digital converter.

15. A detector of emitted photons comprising:
a readout comprising:
a first group of balun transformers being respectively fed by each of the LVDS timing channels, each of the balun transformers in the first group being configured to receive LVDS timing signals from the respective timing channel and convert the received LVDS timing signal to a single-ended output voltage signal; and
one or more second balun transformers configured to convert the single ended output signals from the first group of balun transformers to differential mode output signals; and
a comparator or digitizer configured to receive the differential mode output signals and produce a multiplexed differential timing signal along a multiplexed output channel;
detectors for receiving photons emitted from a patient and to generate timing and energy signals in response, the detectors being arranged in detector arrays; and
a plurality of timing channels, each being respectively coupled to one of the detector arrays and generating one of the LVDS timing signals.

16. The detector of claim 15, wherein each of the detectors comprises:
a scintillator configured to receive photons emitted from a patient; and
a photosensor configured to receive scintillation light from the scintillator in response to the scintillator receiving and to generate timing and energy signals in response.

17. An imaging system comprising:
one or more detector units, each detector unit comprising the detector of claim 15;
processing electronics configured to receive multiplexed timing signals from the readout and to receive the energy signals from the one or more detector units; and
a processor configured to generate an image by processing the received signals.

18. The imaging system of claim 17, wherein the photons emitted from the patient are 511 keV photons.

19. A method for multiplexing a plurality of LVDS fast timing signals from a plurality of timing channels, the method comprising:
for each of the timing channels, receiving the LVDS fast timing signals from the respective timing channel and converting the received LVDS fast timing signals to a single-ended output signal;
converting the single ended output signals to one or more differential mode output signals; and
digitizing the combined differential mode outputs to produce a multiplexed LVDS timing signal along a multiplexed output channel.

20. The method of claim 20, further comprising:
receiving, by each of the plurality of timing channels, a plurality of fast outputs from a set of coupled fast timing sensors;
amplifying the received fast outputs using a low noise RF amplifier;
transforming the amplified fast outputs to differential signals using a balun transformer; and
digitizing the transformed fast outputs to generate the LVDS timing signals for the respective timing channel.
